(19) United States
(12) Patent Application Publication
(10) Pub. No.: US 2011/0275903 A1
(43) Pub. Date: Nov. 10, 2011

(54) Surgical Eye Drape And Method Of Use Thereof

(75) Inventor: Wayne Shelton, Springfield, MO (US)

(73) Assignee: ST. JOHN'S MEDICAL RESEARCH INSTITUTE, INC., Springfield, MO (US)

(21) Appl. No.: 13/100,845

(22) Filed: May 4, 2011

Related U.S. Application Data
(60) Provisional application No. 61/331,928, filed on May 6, 2010.

(51) Int. Cl. A61B 19/08; A61B 1/32

(52) U.S. Cl. 600/236; 128/849

(57) ABSTRACT

A surgical eye drape with a first and second drape sections, each drape section having a wing extending from each edge with a flap for forming a pouch disposed between the wings. The drape sections are disposed above and below an eye with the wings position on opposing sides of the eye and the pouches disposed across the upper and lower eyelids of the eye. The pouches are positioned under the eyelids and the blades of a speculum inserted into the pouches when actuated wrap the drape material around the eyelids which prevents contamination of the eye from bacteria washed from the eyelids during surgical procedures.

### CROSS-REFERENCE TO RELATED APPLICATIONS

This non-provisional application claims priority of U.S. Provisional Patent Application No. 61/331,928 filed on May 6, 2010, which application is incorporated herein by reference.

### BACKGROUND OF THE INVENTION

Complications may arise after eye surgery as a result of intraocular infection associated with the surgery. Despite the use of pre-operative antibiotics, pre-operative routines and sterile draping procedures such infections occur in some patients, with consequences that may be catastrophic.

One source of such intraocular infections is bacteria found on the eyelids of all humans. The eyelids, with the numerous lashes, glands and other skin features are difficult to clean effectively prior to surgery, and even if cleaned continue to present a source of bacteria during the surgical procedure.

This source of bacteria is especially problematic during eye surgery. The eye must be continually irrigated to prevent drying during surgery. The irrigating fluid may wash bacteria from the eyelids into the eye during surgery.

Several eye drapes have been developed to prevent the contamination of the eye during surgery and to limit the potential transfer of bacteria from the eyelid to the eye itself. These drapes are often difficult to use, and often fail, inadvertently exposing the eye to the lid during surgery.

### SUMMARY OF THE INVENTION

The surgical eye drape described herein is simple to use and effective in preventing contact between the eye and the eyelid during surgical procedures. The surgical eye drape comprises a first drape section and a second drape section, each having a first edge. A first wing and a second wing are provided extending from the first edge of the drape sections, with a flap extending from the first edge between the wings, in both drape sections. It is preferred that the flap be folded to form a pouch into which a blade of a speculum is positioned and where the blade is substantially covered by the material forming the pouch.

A method of using the eye drape is also described comprising: providing a first drape section and second drape section, each with two wings extending from a first edge of the drape section, and a flap extending from said first edge between the wings formable into a pouch; disposing the first drape section below the eye with the wings positioned on opposing sides of the eye with the pouch formed therein extending over the lower eyelid; disposing the second drape section above the eye with the wings disposed on opposing sides of the eye and the pouch extending over the upper eyelid; providing a speculum with two blades for securing the eyelids of the eye with one blade adjacent to the upper eyelid and the other blade adjacent to the lower eyelid; positioning each pouch underneath the adjacent eyelid, inserting a blade of the speculum into each pouch and releasing the speculum which allows the blades to separate and physically retain each eyelid in a retracted position.

### BRIEF DESCRIPTION OF THE DRAWINGS

FIG. 1 is a top view of an embodiment of the first and second drape sections of the surgical eye drape in use upon a patient.

FIG. 2 is a detailed top view of an embodiment of the surgical eye drape in use on an eye.

FIG. 3 is a cross sectional view of an embodiment of the surgical eye drape in use on an eye.

FIG. 4 is a detailed perspective view of an embodiment of the surgical eye drape in contact with the upper eyelid of a patient.

FIG. 5 is a top view of an embodiment of the surgical eye drape in position prior to installation of the pouches under the eyelids of a patient.

FIG. 6 is a cross-sectional view of an embodiment of the surgical eye drape showing the user positioning the pouches for use.

FIG. 7 is a cross-sectional view of an embodiment of the surgical eye drape with the pouches in proper position for use.

FIG. 8 is a cross-sectional view of an embodiment of the surgical eye drape showing the blades of a speculum properly positioned within the pouches for use on a patient.

FIG. 9 is a cross-sectional view of an embodiment of the surgical eye drape with the blades of the speculum retracting the patient's eyelids.

FIG. 10 is a top view of an embodiment of a drape section of the surgical eye drape with the flap in a flat configuration.

FIG. 11 is a top view of an embodiment of a drape section of the surgical eye drape with the flap folded to form a pouch.

### DETAILED DESCRIPTION OF THE INVENTION

The surgical eye drape described herein is a simple and easy to use, inexpensive to produce, compatible with most types of ocular surgery, and effective in limiting contamination of the eye during surgery. It is specifically designed to prevent the contamination of the eye with bacteria from the eyelids and other areas of the face surrounding the eye, thus reducing the likelihood of post-operative intraocular infection.

In the embodiment shown in the figures, the surgical eye drape is formed from two sections of flexible material, typically a thin plastic film. The sections may be formed from any material suitable for use as a surgical drape, and that is non-toxic, flexible, capable of sterilization, susceptible to adhesion to itself and to human skin with appropriate adhesives, and has other characteristics typically required for surgical drapes, and specifically surgical eye drapes.

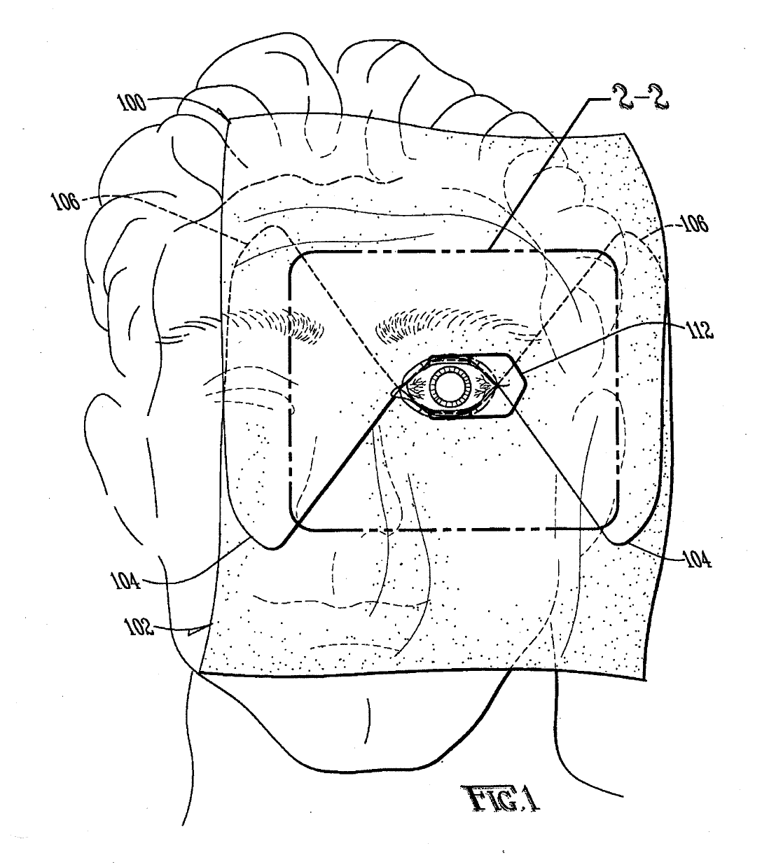

Referring now to FIG. 1, a top view of an embodiment of the surgical eye drape depicts the two drape sections of the surgical eye drape in use on a patient. A first drape section 100 and a second drape section 102 are shown creating a barrier to contamination around the eye. In the embodiment shown in the figures, the drape sections are substantially rectangular in shape with two substantially triangular wings extending on one edge of each drape sections, though other shapes may be utilized so long as the shape of the sections and the wings provides sufficient coverage to prevent contamination of the eye with bacteria and other material from the surface of the patient's head and face around the eye.

The drape sections 100 and 102 shown in FIG. 1 each have wings 104 and 106 extending from along one edge of the drape sections, respectively. The wings 104 and 106 form a notch 108, or area cutout from the edge. In the embodiment shown in the figures, the notches 108 are substantially triangular, though other shapes might be utilized so long as the shape may be formed to the shape of the perimeter of the eye. The notches 108 are shaped to closely surround either the top or bottom lid of a patient's eye when in use. The wings 104 and 106 overlap during use, forming a continuous barrier around the patient's eye.

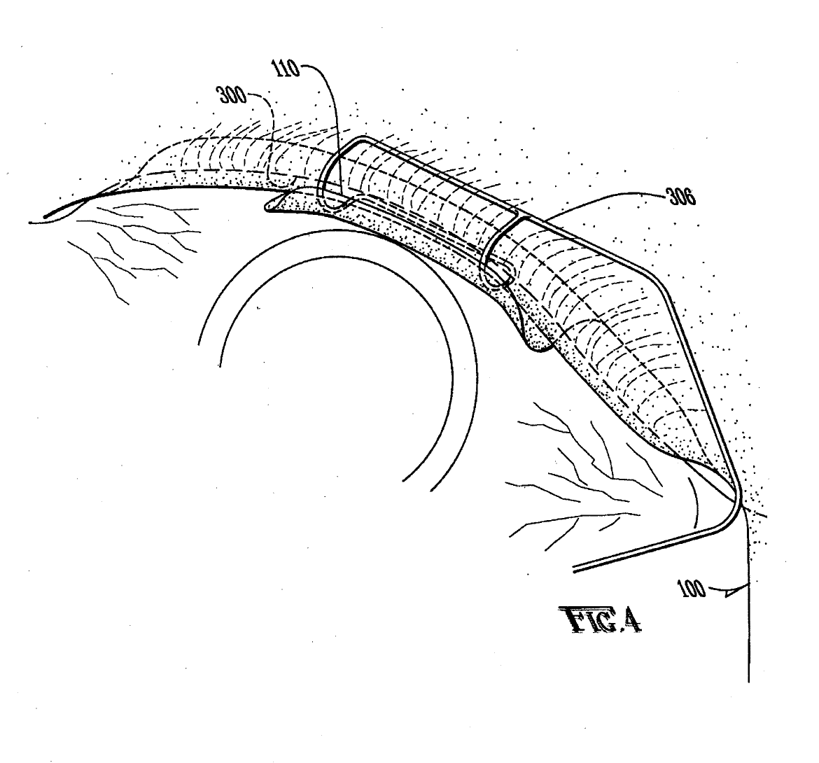

As best shown in FIGS. 4, 5, 10 and 11 and described in relation thereto, each drape section 100 and 102 has a pouch 110 formed from an outer flap 1112 and an inner flap 1114, where the outer flap 1112 is folded onto the inner flap 1114 along a substantially medial line, and the flap margins form a first end and a second end, both of which are substantially closed by an adhesive 1004 which holds the outer flap 112 and inner flap 1114 together to form the pouch 11. The pouch 110 extends from the edge of the drape section 100 or 102 at the area of the notch 108. The pouch 110 is substantially rectangular, but may also narrow from the base of the pouch to the end of the pouch as shown in later figures.

The pouch 110 may be formed into the drape edge, or in some embodiments it may be manufactured as a flap that is folded back to form the pouch by adhering the edges of the flap to the drape section. A retaining clip or speculum 112 is provided for securing the pouch 110 around the eyelids of the patient, and for retracting the patient's eyelids during treatment.

In the embodiment depicted in FIG. 1, the two drape sections are substantially identical in shape and configuration of pouches and adhesive. However, in other embodiments, the two drape sections may be different in shape and configuration, with one section configured for use above the eye and another configured for use below the eye.

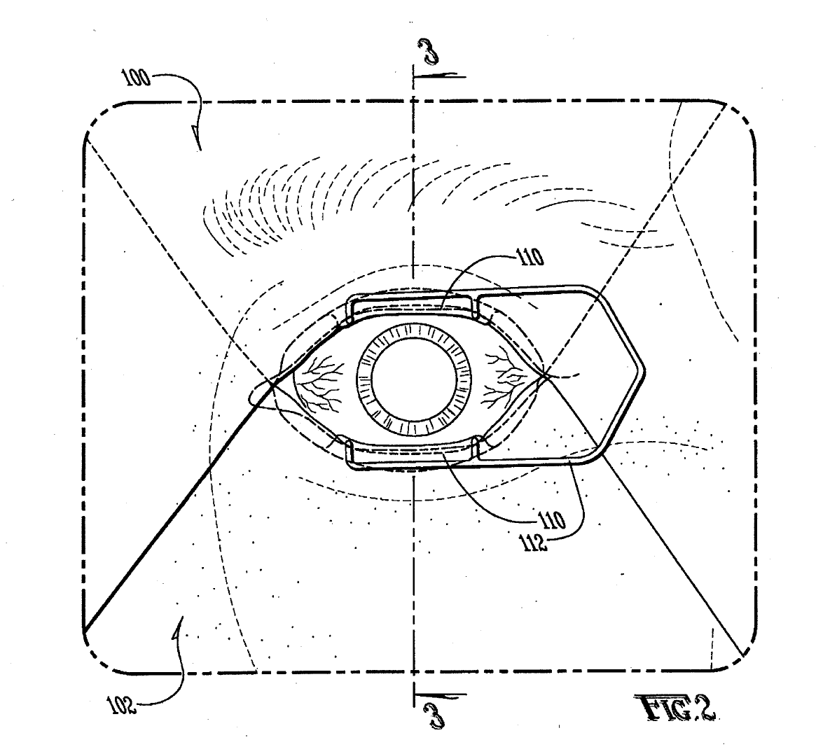

Referring now to FIG. 2, a detailed view of the area labeled 2-2 in FIG. 1 is depicted. The first drape section 100 and second drape section 102 are shown disposed on the face of the patent, overlapping with the notches 108 disposed around the edges of the patient's eye. The speculum 112 is inserted in the eye with one blade under each eyelid. Pouches 110 are wrapped around the speculum blades and run from the drape sections 100 and 102, over the eyelashes and down into the cul-de-sac of the eye, separating the eyelid from the speculum. The pouches 110 then extend back out of the cul-de-sac of the eye and substantially over the opposite side of the speculum blade.

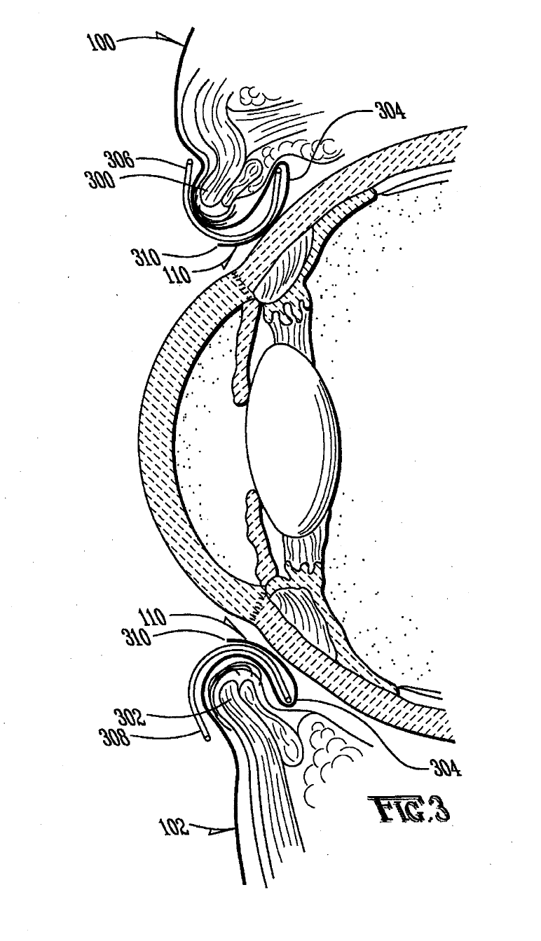

Referring now to FIG. 3, a cross-sectional view along the axis labeled 3-3 in FIG. 2 of an embodiment of the surgical drape is depicted showing the drape in use on a patient. This view clearly shows the disposition of the pouches 110 when the drape is in use. The drape sections 100 and 102 are disposed on the face and head of the patient. Pouches 110 extend over the eyelids 300 and 302 and down into the cul-de-sac 304 of the eye between the eyelids 300 and 302 and speculum blades 306 and 308. The pouches 110 are then wrapped around the speculum blades 306 and 308 and up out of the eye between the blades and the eye.

As can be seen in FIG. 3, the positioning of the pouches 110 covers the eyelids 300 and 302 and areas surrounding the eye during the surgical procedure. This prevents the irrigation of the eye during the procedure from washing bacteria and other contaminants from those areas into the eye. The surgical eye drape is easy to manufacture using techniques commonly known for preparing sterile drapes, and is easy to use in combination with specula 112 that are well known to eye surgeons.

The extension of the sterile pouches 110 over the eyelids 300 and 302 and down into the cul-de-sac 304 shields the eyelids from the irrigating fluid used to keep the eye moist during the surgical procedure. It also creates a barrier to bacteria located on the eyelid, eyelashes or in the other anatomical features surrounding the eye itself. It is also simple to install and use during the procedure. The bridge of speculum 112 exerts outward pressure thus holding the eyelids in a retracted position during use.

Referring now to FIG. 4, a detailed perspective view of one of the drapes in place on a patient's eye is depicted. Drape section 100 is positioned adjacent to the patient's eye with pouch 110 folded over the eyelid 300 and down into the cul-de-sac 304 of the eye. Speculum blade 306 is curved to hold pouch 110 in place in the cul-de-sac and to securely remain on the eyelid during the operation. The outward force of the speculum blade partially folds the drape edge over the eyelid margin as shown.

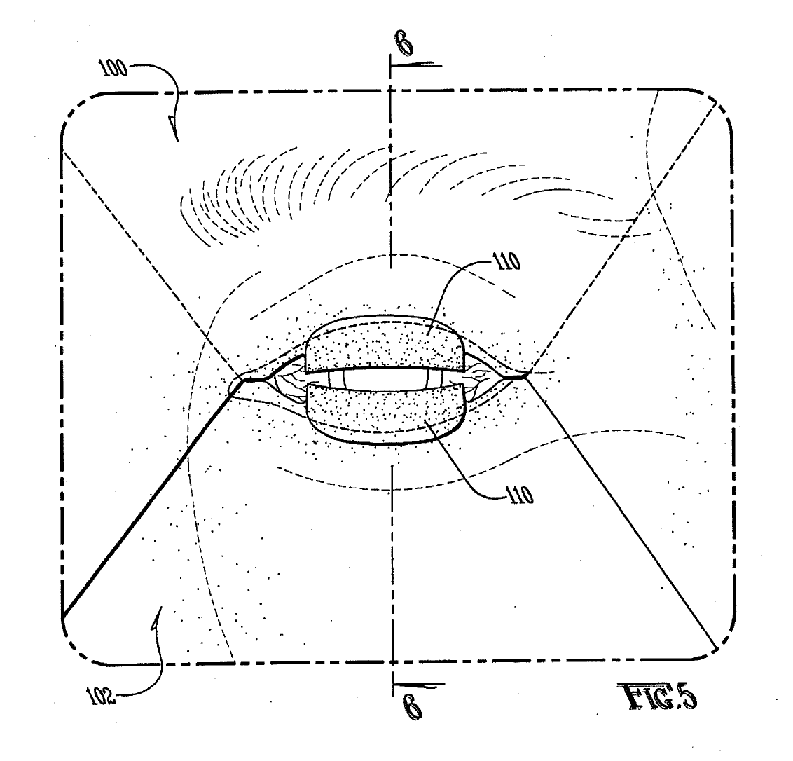

Referring now to FIG. 5, in preparation for use of the surgical eye drape, the two drape sections 100 and 102 are disposed above and below the eye of the patient by the surgeon. The circular notches 108 are disposed around the perimeter of the eye, with the wings 104 and 106 of the two drape sections overlapping to provide a continuous barrier between the patient's face and eyelids and the patient's eye. As will be described in relation to a later figure, in some embodiments adhesives are provided on the drape sections to adhere them to the patient's face and to each other, thus securing them in place. The overlapping drape sections 100 and 102 in this configuration form a sheet with an opening in the middle thereof disposed around the eye of the patient. The pouch 110 on the drape section above the eye extends down across the upper eyelid, and the pouch 110 on the drape section below the eye extends up across the lower eyelid.

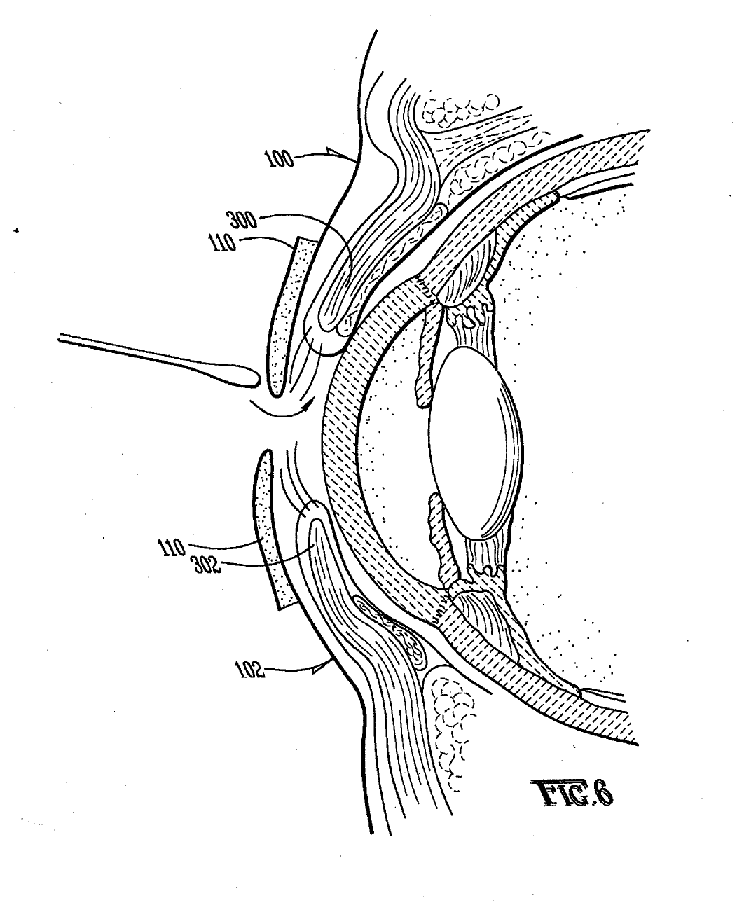

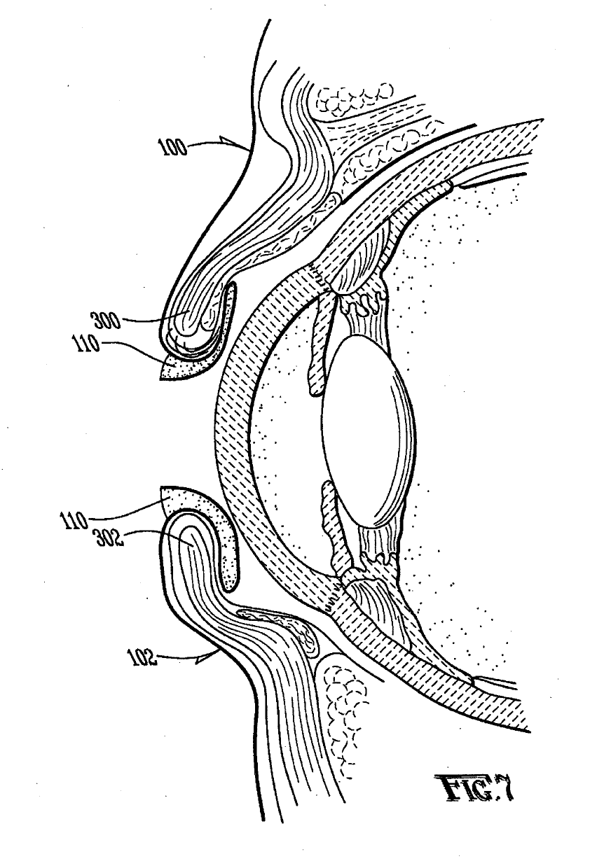

As shown in FIG. 6, the surgeon then inserts the pouches 110 under the eyelids using a cotton swab, their fingers or other appropriate instruments. After the pouches are positioned substantially under the eyelids as shown in FIG. 7, the blades 306, 308 of the speculum 112 are manipulated into the closest associated pouch 110 using common techniques known for manipulating a speculum blade under an eyelid. The surgeon must ensure that the speculum blade is positioned within the pouch prior to releasing the speculum.

The speculum used with the surgical eye drape described herein is a type commonly known for use in eye surgery. Such specula typically have two blades for retaining the eyelids in the open position, and a spring mechanism for allowing the blades to be retracted together, inserted under the eyelids, and then released to the open position. The blades of the speculum may be “open” blades formed from loops of wire, or solid blades formed of a curved sheet of material.

When using the speculum 112 with the surgical eye drape described herein, the blades of the speculum are covered by the pouches 110 extending from the drape sections 100 and 102 as shown in FIG. 5. The pouches 110 are positioned substantially under the associated eyelid and then the blades 306 and 308 of the speculum 112 are positioned into the pouches 110. The pouch is disposed between the blades of the speculum 112 and the surface of the eye. The user of the device applies pressure to speculum 112 to retract the blades 306 and 308 to position 500 to allow the blades to enter below the eyelids 300 and 302. Once the tip of the blades 306 and 308 of the speculum 112, are positioned within pouches 110, and are under the eyelids, the pressure is removed by the user and speculum 112 returns to position 502, holding pouches 110 in place over the eyelids and retracting the eyelids away from the eye itself.

The process of insertion of the speculum 112 is shown in more detail in FIGS. 6-9 which depict a cross-sectional view of the eye along axis 6-6 shown in FIG. 5. In FIG. 6, drape sections 100 and 102 are depicted disposed around the eye, and pouches 110 are being inserted underneath the eyelids with a cotton swab. Eyelids 300 and 302 are still in their natural position and the speculum 112 has not yet been inserted.

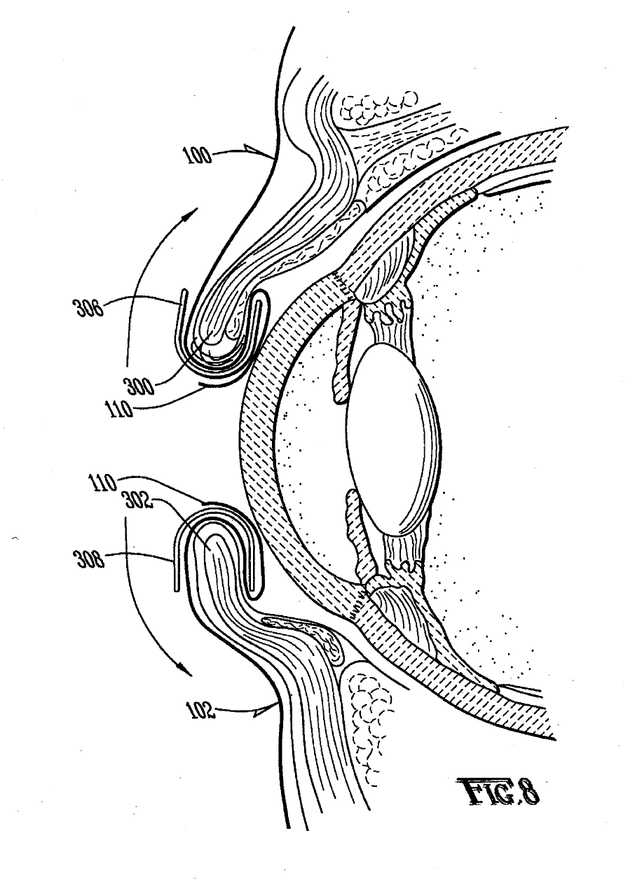

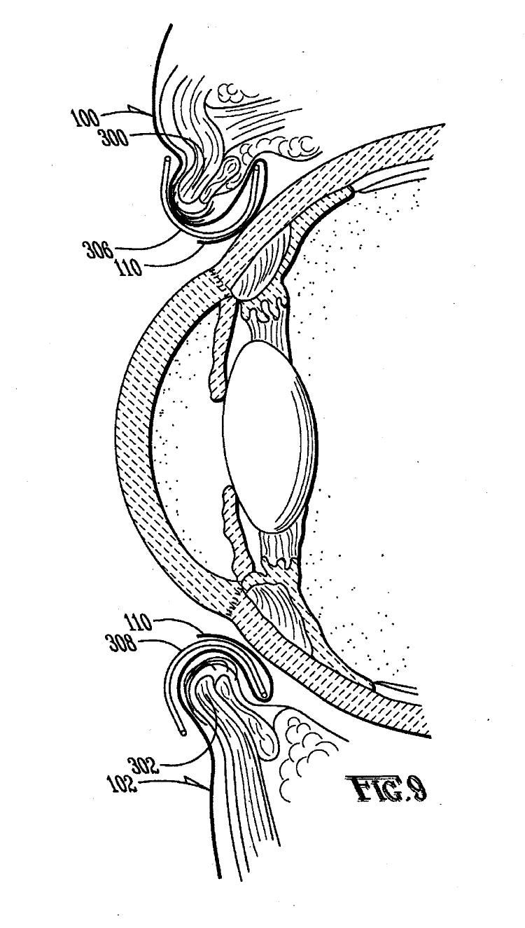

Referring now to FIG. 7, the material of pouches 110 are positioned substantially underneath the eyelids and as shown in FIG. 8, the blades 306 and 308 of speculum 112 are then inserted into the pouches 110 and under eyelids 300 and 302. FIG. 9 depicts the final fully retracted configuration of the eye drape, speculum and eyelids.

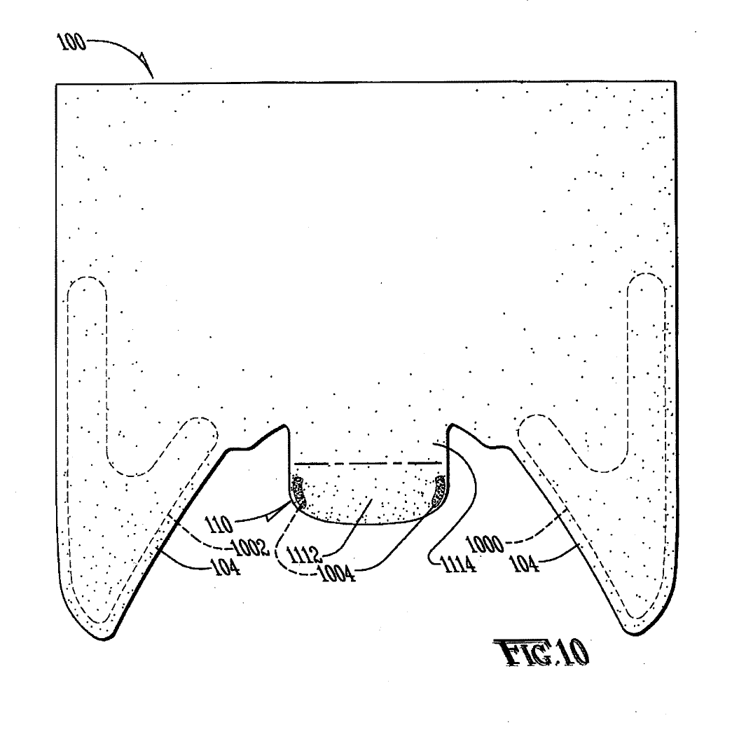

Referring now to FIG. 10, one drape section 100 or 102 is shown in a flat configuration. In an embodiment of the drape, adhesive areas 1000 and 1002 are provided on the bottom surface of wings 104 and extending along the edge of the drape section 100. These adhesive areas allow the drape section to be securely attached to the face of the patient and to the other drape section. This prevents the drape from shifting or folding during use. The adhesive may be disposed in other areas of the drape section, or may be replaced with a separate adhesive, tape or some other method of temporarily securing the drape to the patient's face, without limiting the scope of the invention.

The drape section 100 is depicted with pouch 110 in an unfolded configuration comprising a flap. As shown, the provided flat flap has an outer portion 1112 and an inner portion 1114 and a margin therebetween. The outer portion 1112 is folded at the margin back onto the inner portion 1114 of the flap and secured in place by the user. In the depicted embodiment, adhesive areas 1004 are disposed on the top surface of the inner flap for adhering the outer portion of the flap to the drape section, which is folded on the margin. In other embodiments, the pouch may be formed by heat seal, or other means of attaching or bonding two sheets of material.

The pouch is preferably formed from the flap during manufacturing but it is understood that in some instances, it may be desirable for the user to use the flat flap to form a pouch having a specific size or purpose to accommodate, for instance different installation methods or for equipment compatibility.

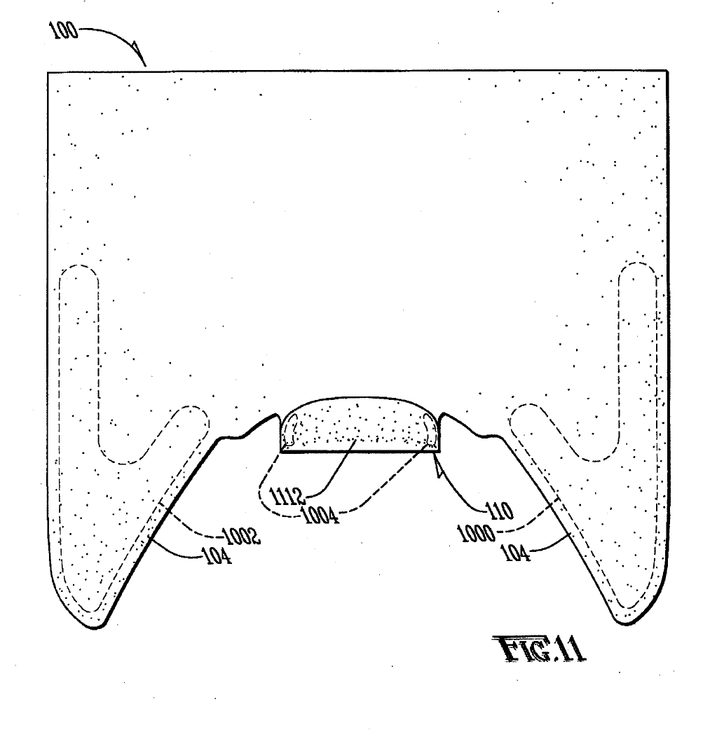

Referring now to FIG. 11, Pouch 110 is shown in the preferred folded configuration, and it has been secured in the folded position by adhesive areas 1004 or other bonding means which may occur during manufacturing or by the user during use of the drape. In various embodiments of the eye drape the pouch may be manufactured in a folded and adhered configuration.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

What is claimed is:

1. A surgical eye drape for use with a speculum comprising:
a first drape section and a second drape section each having a first wing and a second wing extending from a first edge of each drape section; and
a flap disposed between the first and second wings in each of the first and second drape sections.

2. The surgical eye drape of claim 1 wherein a first and second edge of each flap are bonded to the drape section forming a pouch with an open edge disposed toward the drape section.

3. The surgical eye drape of claim 2 further comprising adhesive disposed on the first and second drape sections to adhere the sections to a patient and to each other.

4. The surgical eye drape of claim 3 wherein the first and second wings are substantially triangular.

5. The surgical eye drape of claim 4 wherein a first and second blade of the speculum may be inserted into the pouch on each drape section.

6. A method of draping an eye for a surgical procedure, comprising the steps of:
providing a first drape section and a second drape section, each drape section having a first wing and a second wing extending from a first edge of each drape section, and a flap disposed between the wings;
disposing the first drape section adjacent to the eye with the first and second wings positioned on opposing sides of the eye and with the flap extending over a first eyelid;
disposing the second drape section adjacent to the eye with the first and second wings positioned on opposing sides of the eye and overlapping the first and second wings of the first drape section, and the flap extending over a second eyelid;
providing a speculum with two blades for securing the eyelids of the eye with one blade adjacent to the upper eyelid and the other adjacent to the lower eyelid;
forming each flap into a pouch for receiving a blade of the speculum;
positioning one pouch around each eyelid;
positioning each blade of the speculum into one of the pouches; and
releasing the speculum such that the blades are urged apart to forcibly separate and retain the eyelids.

7. The method of claim 6 wherein the step of forming each flap into a pouch for receiving a blade of the speculum comprises the steps of:
folding the flap so that a first edge of the flap and a second edge of the flap, opposed to the first edge, are in contact with the drape section; and
bonding the first and second edges of the flap to the drape section.

8. The method of claim 7 wherein the step of bonding the first and second edges of the flap to the drape section further comprises the steps of providing adhesive adjacent to the first and second edges of the flap.

9. The method of claim 7 wherein the step of bonding the first and second edges of the flap to the drape section further comprises the steps of heat bonding the areas adjacent to the first and second edges of the flap to the drape section.

10. The method of claim 7 wherein the step of positioning each blade of the speculum into one of the pouches further comprises the steps of:
retracting the blades of the speculum;
inserting each blade into one of the pouches;
positioning the pouch and the speculum in the cul-de-sac of the eye under the adjacent eyelid.
